(19) United States
(12) Patent Application Publication
(10) Pub. No.: US 2008/0063732 A1
(43) Pub. Date: Mar. 13, 2008

(54) Method of increasing the efficacy of neurotoxin

(76) Inventor: Basil M. Hantash, E. Palo Alto, CA (US)

Correspondence Address: LUMEN PATENT FIRM, INC., 2345 YALE STREET, SECOND FLOOR, PALO ALTO, CA, 94306, US

(21) Appl. No.: 11/888,660

(22) Filed: Jul. 31, 2007

Related U.S. Application Data
(60) Provisional application No. 60/834,590, filed on Jul. 31, 2006.

(51) Int. Cl. A61K 35/74; A61K 33/06; A61K 36/00; A61P 21/00; A61P 43/00

(52) U.S. Cl. 424/685; 424/195.18; 424/780

(57) ABSTRACT

The present invention provides a method of increasing the efficacy of treating a muscle or sweat gland hyperactivity condition in a human or mammal with a neurotoxin. The method includes adding a quantity of either a diffusion-limiting agent or an anti-metabolic agent to the neurotoxin. Adding the agent to the neurotoxin leads to the formation of an agent/neurotoxin injection mixture. The quantity of agent added is sufficient to increase a therapeutic response of the human or mammal's sweat gland or muscle to the neurotoxin compared to what the response would have been had the human or mammal been treated with neurotoxin alone. The present invention provides a kit for treating a muscle or sweat gland hyperactivity condition in a human or mammal. The kit includes a needle, a solvent, a neurotoxin, and either a diffusion-limiting agent or an anti-metabolic agent.

### CROSS-REFERENCE TO RELATED APPLICATIONS

This application claims priority from U.S. Provisional Patent Application No. 60/834,590, filed Jul. 31, 2006, which is incorporated herein by reference.

### FIELD OF THE INVENTION

The present invention relates generally to medical and cosmetic treatment. More particularly, the present invention relates to a method for increasing the efficacy of neurotoxin in the treatment of humans and animals.

### BACKGROUND

Relaxation of hyperactive muscles or sweat glands by injection of neurotoxin represents one of the most common procedures performed in the United States. For example, botulinum toxin has been used on millions of patients, and its safety and efficacy has been well established. Notwithstanding its impressive safety profile, some 3.2% of patients experience blepharoptosis, the most common adverse event. More recently, however, injection of botulinum toxin has led to respiratory arrest and death in several patients. In addition, botulinum toxin is expensive. Accordingly, there is a need in the art to develop methods of increasing the efficacy and safety of neurotoxins to reduce the amount of neurotoxin needed for a given procedure.

### SUMMARY OF THE INVENTION

In one embodiment, the present invention provides a method of increasing the efficacy of treating a muscle or sweat gland hyperactivity condition in a human or mammal with a neurotoxin. The method includes adding a quantity of either a diffusion-limiting agent or an anti-metabolic agent to the neurotoxin. Adding the agent to the neurotoxin leads to the formation of an agent/neurotoxin injection mixture. The quantity of agent added is sufficient to increase a therapeutic response of the human or mammal's sweat gland or muscle to the neurotoxin compared to what the response would have been had the human or mammal been treated with neurotoxin alone.

In another embodiment, the present invention provides a kit for treating a muscle or sweat gland hyperactivity condition in a human or mammal. The kit includes a needle, a solvent, a neurotoxin, and either a diffusion-limiting agent or an anti-metabolic agent.

### BRIEF DESCRIPTION OF THE FIGURES

The present invention together with its objectives and advantages will be understood by reading the following description in conjunction with the drawings, in which:

FIGS. 1-3 show examples of results of improved efficacy of botulinum toxin upon addition of a diffusion-limiting agent according to the present invention.

### DETAILED DESCRIPTION OF THE INVENTION

According to the method of the present invention, a quantity of a diffusion-limiting agent or an anti-metabolic agent is added to a neurotoxin to form an agent/neurotoxin injection mixture. This may be accomplished in several ways. In one aspect of this embodiment, a lyophilized form of the agent is added to a lyophilized form of the neurotoxin to make the injection mixture. This could occur, e.g., as part of the manufacturing process. The dry mixture is then reconstituted to the appropriate concentration. In another aspect of this embodiment, a liquid form of the agent is added to a lyophilized form of the neurotoxin, e.g. by the end-user. In yet another aspect of this embodiment, a liquid form of the agent is added to a liquid form of the neurotoxin. For example, an end-user could take what is needed from a vial of neurotoxin and add the appropriate amount of liquid agent to it.

Any neurotoxin may be used according to the present invention. Preferably, the neurotoxin is a paralytic agent and/or an anti-hidrotic agent. Preferred paralytic agents include, but are not limited to, tetanus toxin, omega-conotoxin, curare, and botulinum toxin. Any type of botulinum toxin may be used according to the present invention, including but not limited to types A, B, C, D, E, F, and G. Similarly, any anti-hidrotic agent may be used according to the present invention. Examples include, but are not limited to, botulinum toxin, aluminum chloride, glycopyrrolate, and robinol.

The amount of neurotoxin used will depend on the condition to be treated. In addition, even within a given neurotoxin, different preparations differ. For example, the preparations of botulinum toxin type A (BTX-A) marketed in the United States (BOTOX®, Allergan; Irvine, Calif.), the United Kingdom and Europe (Dysport by Speywood-Vaccine and Research Laboratory-Porton Down; Salisbury, UK), and Japan (CS-BOT) differ in potency. According to 1 report, 1 nanogram of BTX-A contains approximately 20 U of BOTOX®, or 1 U of BOTOX® is equal to approximately 0.05 nanogram of the toxin. According to another report comparing the 3 different preparations of BTX-A, 1 nanogram of Dysport contains approximately 40 mouse U, whereas 1 nanogram of the BOTOX® contains approximately 4 mouse U, and 1 nanogram of CS-BOT contains approximately 15.2 mouse U. Clinically, one U of BTX-A is approximately equivalent to 3 U of Dysport. Standardization efforts are underway by measuring pharmacologically relevant actions of BTX-A (eg, median paralysis unit).

Botulinum toxin type B is marketed in the United States as MyoBloc. This preparation is a ready-to-use solution that does not require reconstitution available in 3 vial sizes (ie, 2500 U, 5000 U, and 10,000 U), and it is stable for up to 21 months in refrigerator storage.

Botulinum toxin has been used clinically for many different conditions, including focal dystonias, spasticity, non-dystonic disorders of involuntary muscle activity, eye disorders, disorders of localized muscle spasms and pain, smooth muscle hyperactive disorders, and cosmetic use.

For example, use of BTX-A in different types of focal dystonias has been studied well and has proven to be very effective. Botulinum toxin injection is the treatment of choice for cervical dystonia (spasmodic torticollis). This injection benefits the highest percentage of patients in the shortest time and has been proven effective in many double-blinded placebo-controlled trials. Botulinum toxin injection has fewer side effects than other pharmacological treatments.

For focal dystonias, treatment dosages of BTX-A in the United States have been reported to range from 100-300 U per patient (BOTOX). One hundred U of toxin per mL of preservative-free normal saline are used commonly.

Injections for focal dystonias are performed with a Teflon-coated 24-gauge needle connected to an electromyographic (EMG) machine. Those muscles with highest clinical and EMG activity are injected. Usually in one session, 2-4 separate muscles are injected and, in larger muscles, 2-4 sites per muscle are injected.

No general consensus exists among users of botulinum toxin regarding the need for EMG guidance while injecting botulinum toxin for cervical dystonia. EMG guidance, however, is helpful, particularly in obese patients whose neck muscles cannot be palpated adequately.

Identifying the specific muscles involved in cervical dystonia prior to the injection is important. The sternocleidomastoid, trapezius, splenius capitis, and levator scapulae muscles are injected most commonly.

Beneficial effect from toxin injection usually is apparent in 7-10 days. Maximum response from the toxin is reached in approximately 4-6 weeks and lasts for an average of 12 weeks. Injections usually are repeated every 3-4 months.

Performing BOTOX® injections to treat horizontal forehead lines is relatively easy, and the result usually is quite satisfying. Treatment can include injections for glabellar frown lines when appropriate. The frontalis muscle elevates the eyebrows and the skin of the forehead. The fibers of the frontalis are oriented vertically, and wrinkles of the forehead are oriented horizontally. The frontalis muscle originates on the galea aponeurotica near the coronal suture and inserts on the superciliary ridge of the frontal bone and skin of the brow, interdigitating with fibers of the brow depressors (ie, procerus, corrugator supercilii, orbicularis oculi muscle). The medial fibers usually are more fibrous than the lateral fibers, thus requiring less toxin for paralysis. Total paralysis of the frontalis must be avoided, since this is likely to cause brow ptosis and loss of expression. Injection too close to the lateral eyebrow can cause lateral eyebrow ptosis.

Multiple injections of small amounts of toxin create weakness without total paralysis. Typically, one injects 3-5 sites on each side of the mid line, usually using 2 units (1-3 U) per site. Sites are separated by 1-2 cm. An initial injection site is chosen approximately 1 cm above the eyebrow vertical to the medial canthus. Additional sites diverge laterally and upward to the hairline in a “V” configuration, often for a total of 3 sites. Additional sites (1-3) can be added in the mid line or more laterally (1-2) depending on individual and clinical response.

If wrinkles extend to the temporal region, lateral injections can be performed. Caution must be used to prevent injecting lateral to the lateral canthus to avoid inhibiting temporalis function. Caution must also be used when injecting patients in whom the hyperfunctional frontal lines support a ptotic upper eyelid.

Injections of the upper face and periocular region usually are performed with the patient seated, and the patient is asked to remain upright for 2-3 hours to prevent spread of toxin through the orbital septum.

Glabellar frown lines are the most common reason for cosmetic injection of BOTOX®. Facial rhytids and folds in this area result from action of the depressor muscles (ie, corrugator supercilii, depressor supercilii, orbicularis oculi, procerus). The corrugator superciliaris, medial orbital portion of the orbicularis oculi, and more vertically oriented fibers of the depressor supercilii produce the vertical lines of the glabella.

Usually, 5 sites are injected with 4-6 units each for an average total dose of approximately 25 units. One site on each side is used to inject the corrugator, one site on each side is used to inject the orbicularis oculi and depressor supercilii, and one site is used to inject the procerus in the mid line.

The patient is asked initially to frown and scowl, and the target muscles are palpated. The first injection is placed into the belly of the corrugator muscle. The needle is inserted at the origin of the corrugator fibers just above the medial canthus and superciliary arch until bone is felt, and then withdrawn slightly. The needle is advanced within the belly of the muscle upward and lateral as far as the medial third of the eyebrow, 1 cm superior to the orbital rim. 4-6 units are injected as the needle is withdrawn.

The next site is approximately 1 cm above the upper medial aspect of the supraorbital ridge. The needle is advanced slightly in a vertical direction toward the hairline. 4-6 units are injected into the orbicularis oculi and depressor supercilii as the needle is withdrawn. These injections are repeated on the contralateral side.

The last injection is placed into the belly of the procerus to eliminate the horizontal lines at the root of the nose. 4-6 units are injected at a point where 2 lines drawn at 45° from the medial aspect of the eyebrows converge in the center of the nasal root, just superior to the horizontal plane of the medial canthi. To avoid resultant accentuation of eyebrow arching in men, an additional 4-6 units are injected 1 cm above the supraorbital prominence vertical to the mid point of the eyebrow.

An effective starting dose is typically from 2.5-4 units per injection site (12.5-20 U total).

A glabellar “spread test” may be performed prior to injection by spreading the glabellar wrinkles apart with the thumb and index fingers. This may allow an estimate of the expected benefit from BOTOX® injection. Patients with thick sebaceous skin and deep dermal scarring that are not improved with manual spreading usually respond poorly to botulinum toxin injections. EMG guidance may provide valuable information when initial injections prove unsatisfactory.

Aging and photodamage cause much of the wrinkling in the lateral canthal area. However, the component of hyperfunctional contraction of the lateral aspect of the orbicularis oculi is targeted for improvement with BOTOX® injections. The lateral fibers of the orbicularis oculi are arranged in a circular pattern around the eye. Contraction of these fibers produces wrinkles that extend radially from the region of the lateral canthus.

Three or 4 subcutaneous injections are performed approximately 1 cm lateral to the lateral orbital rim using 2-3 units per injection site (for a total of 6-12 U per side). Sites are spaced 0.5-1 cm apart in a vertical line or slightly curving arch. Doses that are too high or injections that are too medial can lead to eyelid ptosis or diplopia.

With age, the lateral eyebrow typically becomes ptotic before the medial aspect. The lateral brow is more affected by the gravitational descent of the temporal soft tissue mass and the downward force of the corrugator supercilii and orbicularis oculi muscles. Temporal brow lift can be achieved by BOTOX® injections into the lateral brow depressors. Patients seeking elevation in eyebrow height may be treated with 7-10 units of BOTOX® injected into the superolateral portion of the orbicularis oculi below the lateral third of the brow. Injecting superior and lateral to the orbital rim minimizes the potential for ptosis.

Contraction of the levator labia superius muscle is observed when an individual “scrunches” his or her nose, and hyperfunctional contraction often confers a “mad dog” appearance to the face. This muscle can be injected with 2-5 units of BOTOX® on each side of the nose lateral to the nasion.

Undesirable nasal flare may be treated successfully with serial BOTOX® injections.

Reduction of a prominent mental crease can be achieved by injection of 5-10 units of BOTOX® into the point of the chin.

Injections of BOTOX® can improve facial asymmetry and synkinesis. The asymmetry of residual unilateral paresis can be reduced by judicious injection of normal muscles on the unaffected side. Hyperkinesis from aberrant degeneration can be reduced by injection of hyperkinetic muscles.

Botulinum toxin injections may be used to treat upper lip wrinkling in individuals with 2 or 3 deep wrinkles. Small doses of botulinum toxin (0.5-1 U per wrinkle) may be administered, injected superficially rather than deeply. Avoiding weakness of the upper lip is important.

To weaken the depressor anguli oris, which is the underlying muscle of the downturn at the corner of the mouth, 2-3 units of botulinum toxin may be injected. The individual is instructed to forcibly pull down the corners of the mouth; the depressor anguli oris can be felt inferior to a point 1 cm lateral to the commissure.

Low doses (2-3 U) of BOTOX® can be injected in the levator labii superioris alaeque nasi to soften the nasolabial fold. However, achieving a good result in attempting to soften the nasolabial fold is difficult.

BOTOX® injections may be used to provide presurgical chemodenervation of the brow depressor muscles, giving better results with surgical repositioning of the brow. Pretreatment of crow's feet allows the surgeon to better define the incision line within the confines of the bony orbital margin.

Cosmetic BOTOX® injection of the neck generally are considered most appropriate for older patients who are not candidates for surgery, older patients who previously have had neck rejuvenation surgery without relapse or severe neck skin excess, and younger patients with strong platysmal bands who are not yet surgical candidates. They also can be used to improve the cosmetic outcome after rhytidectomy. Younger patients have a noticeably better result, as do older patients who have had prior surgery. Patients exhibit greater improvement during animation than in repose. After preparing the skin, the patient is asked to contract the platysma muscle; this identifies its bands. Injection techniques vary among practitioners. Some use a few injections along the length of a band while others use many. Some practitioners use EMG guidance, although this usually is not necessary. One technique involves injecting each band at 1.0- to 1.5-cm intervals from the jawline to the lower neck. Approximately 3-10 units may be injected, depending on the thickness of the platysmal band.

Another technique involves injecting the bands at the following 3 sites: the curve between the horizontal submental surface and the vertical anterior surface, at points midway between this point and the anterior extent of the band on the submental surface, and the inferior extent of the band on the anterior neck. The 2 large bands can be injected with 20 units each and smaller bands with 5 units each.

Most patients require a total of 50-100 units, and some require as many as 200 units. Caution must be used to inject the platysma muscles and not the muscles beneath them, since this is more likely to cause swallowing weakness.

Tetanus toxin causes a block of neuromuscular transmission. Tetanus toxin may be used at a dose of about 1 μg/ml toxin. Tubocurarine and vecuronium may be used at about 0.1 mg/kg for and 0.01 mg/kg, respectively. The neurotoxic peptide huwentoxin-III may be used at dosage of between about 2 to 200 μg/g.

Any anti-metabolic agent may be used according to the present invention. In a preferred embodiment, the anti-metabolic agent is a collagen or hydrogel. Collagen is preferably in the injection mixture at a concentration in the range of between about 0.3 to about 30 mg/ml, most preferably about 10 mg/ml. Hydrogel is preferably in the injection mixture at a concentration in the range of between about 1 to about 30 mg/ml, most preferably about 10 mg/ml.

Similarly, any diffusion-limiting agent may be used according to the present invention. In a preferred embodiment, the diffusion-limiting agent is a dextran, hyaluranic acid, or epinephrine. Dextran is preferably in the injection mixture at a concentration in the range of between about 50 mM to about 500 mM, most preferably about 250 mM. Hyaluronic acid is preferably in the injection mixture at a concentration in the range of between about 3 to about 100 mg/ml, most preferably about 30 mg/ml. Epinephrine is preferably in the injection mixture at a concentration in the range of about 1 to about 100 μg/ml, most preferably about 10 μg/ml. Other diffusion-limiting agents that can be used instead of epinephrine include norepinephrine, and other alpha-adrenergic receptor agonists. Levonordefrin is preferably used in a range of 6 to 600 μg/ml, but most preferably 60 μg/ml. In patients with a cardiac condition, the dose of epinephrine and levonordefrin is reduced 5-fold for safety reasons. Another diffusion-limiting agent that may be used is ornipressin, which is preferably used at a concentration of between 0.01 IU to 0.1 IU per ml. Other diffusion-limiting agent agents that can be employed include but are not limited to phenylephrine, pseudoephedrine, angiotensin, endothelin, and vasopressin.

Addition of a diffusion-limiting agent or an anti-metabolic agent leads to reduced diffusion of neurotoxin into the lymphatic and capillary systems. Diffusion otherwise would reduce the effective concentration of neurotoxin found at a local synapse. Thus, this leaves more neurotoxin toxin to act at each synapse, helping reduce the quantity required for achieving a particular effect. Reduced blood flow also reduces local activity of metabolic enzymes that may break down the neurotoxin. In addition, epinephrine and other adrenergic agonists can block calcium-dependent exocytosis of neurotransmitter at the presynaptic terminal, thus acting synergistically with the neurotoxin. Furthermore, anti-metabolic agents such as collagen or hydrogel allow for slow release of the neurotoxin from the viscous agent/neurotoxin reconstituted mixture that is injected into the target site. This creates a reservoir of neurotoxin that is slowly released rather than saturating the target site. Avoidance of saturation is useful in certain situations where neurotoxin-mediated receptor desensitization leads to downregulation of the receptor and the initiation of a cascade that eventually replaces the receptor. Thus, slow release of neurotoxin leads to a sustained efficacy and avoids triggering receptor replacement, which restores muscle or sweat gland activity.

Addition of a diffusion-limiting agent or anti-metabolic agent preferably increases efficiency of neurotoxin treatment in at least one of the following ways. In first way, addition of a diffusion-limiting agent or an anti-metabolic agent reduces the amount of neurotoxin needed to treat a given condition by between about 25% to about 75%. Alternatively, or in addition, a diffusion-limiting agent or an anti-metabolic agent preferably accelerates onset to as soon as 1-4 days after injection. Alternatively, or in addition, time to peak efficacy is preferably reduced to as soon as 7-30 days post injection. Finally, the duration of the effect of the neurotoxin is preferably lengthened to up to 6-12 months. As a result, the present invention preferably leads to increased efficacy, accelerated onset and time to peak effect, and prolonged duration with decreased side effects such as eye ptosis and respiratory arrest.

Any muscle hyperactivity condition may be treated according to the present methods, including but not limited to bladder dysfunction, migraine, facial rhytides, and lower lip asymmetries caused by a hyperkinetic depressor labii inferioris. In a preferred embodiment, the condition is periorbital rhytides.

Conditions other than muscle hyperactivity can also be treated according to the present methods, including but not limited to hyperhidrosis of the palms, soles, axilla, and forehead.

To treat the muscle or sweat gland hyperactivity condition, the botulinum toxin/vasoconstrictor combination should be injected in an appropriate site on the body of the human or mammal, as known in the art. For example, for periorbital rhytides, the combination would be injected lateral to the lateral canthus of the human or mammal being treated.

The present invention also provides a kit for treating a muscle or sweat gland hyperactivity condition. The kit includes a needle, a solvent, a neurotoxin, and either a diffusion-limiting agent or an anti-metabolic agent. Details of the neurotoxins, diffusion-limiting agents, and anti-metabolic agents can be found above. The solvent may be any solvent, but is preferably at least one of preservative-free saline, preservative containing saline, water, or metabisulfite. Metabisulfite allows for increased stability of epinephrine, further enhancing its ability to localize neurotoxin. This is by reducing pH to 3.5, where epinephrine is more stable.

According to the present invention, the neurotoxin, agent, or both may be lyophilized or in liquid form. In addition, the neurotoxin and agent may be provided separately or combined into one vial.

### EXAMPLE

Study on the Effect of Epinephrine on Botulinum Toxin Treatment for Periorbital Rhytides

Methods

Study Design

The study was approved by the Stanford University Institutional Review Board and Ethics Committee. All patients were consented prior to treatment. A split-face double-blind randomized study enrolled adult patients over the age of 18 years. Inclusion criteria were no prior use of Botox, presence of bilateral moderate to severe crow's feet at maximum contraction, and age over 18. Exclusion criteria were prior treatment with Botox, unilateral or asymmetric crow's feet, use of medications that interfere with neuromuscular function such as aminoglycoside antibiotics or curare-like agents, and a history of myasthenia gravis or other neuromusclular disorders of the face. Patients were also excluded if they reported a prior proven allergy to Botox, a history of congestive heart failure or multiple myocardial infarctions. The subjects were required to be available for longitudinal study over the course of at least six months in order to assure appropriate post-injection follow-up. The length of follow-up was based on the current literature indicating that duration of effect for first time users of Botox is approximately 3-6 months. Informed consent was obtained from all patients prior to study enrollment.

Treatment Regimen

Fourteen subjects (ages 39-57) with moderate to severe periorbital rhytides were enrolled. Each patient was double-blinded to the two treatment arms, Botox and Botox plus EPI 1:100,000. The experimental side was randomly chosen in consecutive fashion by the staff member mixing the Botox. This person was not involved in the evaluation, maintaining the validity of the double-blinding. The two treatments were freshly prepared with identical concentrations of Botox diluted with preservative-free saline. A 1 ml ampule of EPI 1:1000 (1 mg per ml) pre-dissolved in sterile water with sodium chloride added for isotonicity, and no greater than 0.1% sodium bisulfite as an antioxidant, served as the stock solution. The final volume (0.3 ml containing 12 units of Botox) and concentrations of Botox (40 units per ml) injected were identical for all study subjects. Each side of the face was marked and injected at one site lateral to the lateral canthus. Botox injections were performed by an experienced operator (HBG). The physician (HBG) who injected each patient immediately left the treatment room after injection and did not have an opportunity to observe differences, if any, in blanching of the skin post-injection.

Outcome Measures

The patients were followed prospectively and photographs were taken at 0, 4, 30, 90, and 180 days to assess the clinical effect of the two treatment arms. Patient consent was obtained for clinical photography. Baseline crow's feet were determined by objective scoring using a 0-3 nominal scale, corresponding to no, mild, moderate, or severe periorbital rhytides. Subjective and objective evaluations rating clinical improvement of periorbital rhytides were assessed using a nominal scale from 0-4, corresponding to 0, 1-25%, 26-50%, 51-75%, and 76-100% improvement, respectively. The incidence of adverse events was also recorded. At the first follow-up visit, patients were asked if they could guess which side received the experimental treatment. All patients responded that they could not, confirming the validity of subject blinding. In addition, this physician (HBG) who performed the injection was not involved in any of the follow-up assessments, further assuring the maintenance of double-blinding. At the completion of the study, results were analyzed for onset of action, duration of effect, and incidence of adverse events. The analysis of clinical improvement was conducted on an intention-to-treat basis and included all patients who received initial therapy and attended at least one post-treatment follow-up. Means±standard error of the mean (SEM) were calculated. Bar graphs derived from the raw nominal score data as well as the calculated means were plotted for each treatment and follow-up visit. Non-parametric (Mann-Whitney) and student's t-tests were used to test significance of the data. An alpha level of p<0.05 was interpreted as significant for all analyses.

Results

Demographics

Fourteen subjects were enrolled in the study (Table 1). Ten of 14 subjects (71%) were female. Twelve of 14 patients were Caucasian (86%) with one Hispanic and one Asian subject. The mean age was 49 years with a range of 39 to 57 years. Baseline crow's feet were objectively scored using a 0-3 nominal scale, corresponding to no, mild, moderate, and severe periorbital rhytides. All patients fell between moderate to severe with a mean baseline score of 2.4 for both the left and right side (data not shown); this was expected since the study restricted enrollment only to those with symmetric crow's feet.

Efficacy

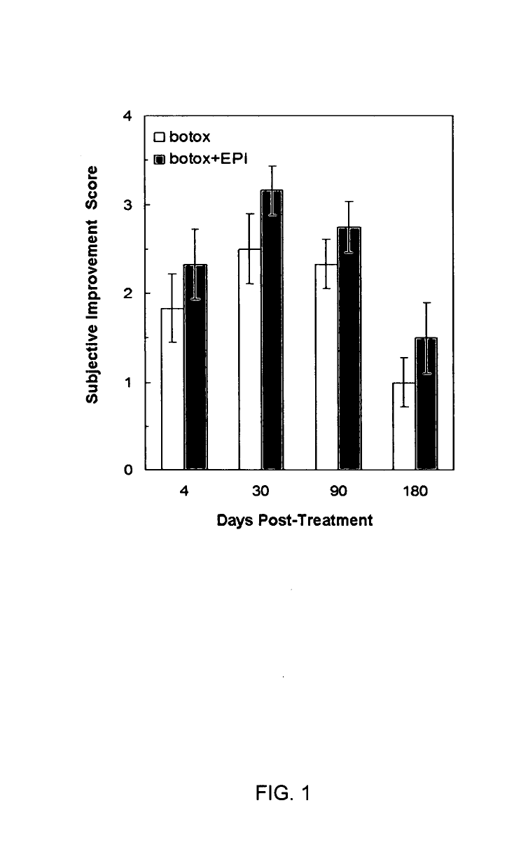

Of the 14 enrolled patients, 13 patients completed the 6 month study (Table 1). Of these patients, 12 were responders and only one did not appear to respond to Botox (12 units in 0.3 ml per side). One patient was withdrawn from the study after receiving asymmetric doses of Botox. Patients were asked to rate the clinical improvement of periorbital rhytides using a nominal scale of 0-4, corresponding to 0, 1-25%, 26-50%, 51-75%, and 76-100% improvement, respectively. FIG. 1 shows the subjective patient scores across the 6-month study period examined for the 12 responders. Within 4 days after treatment, 50% of responders ( 6/12) reported a significant difference in the improvement of dynamic lines between the two sides. The mean for Botox was 1.8 versus 2.3 for Botox plus EPI (p<0.01).

At the 1 month mark, 75% of responders ( 8/12) noted a significant difference between the two sides with a mean score of 2.5 for Botox versus 3.2 for Botox plus EPI (p<0.001). By three months, the mean effect of Botox (2.3) and Botox plus EPI (2.8) had peaked and fallen slightly, although this difference (p<0.02) remained statistically significant even six months after treatment (mean of 1.0 versus 1.5 for Botox and Botox plus EPI, respectively; p<0.005).

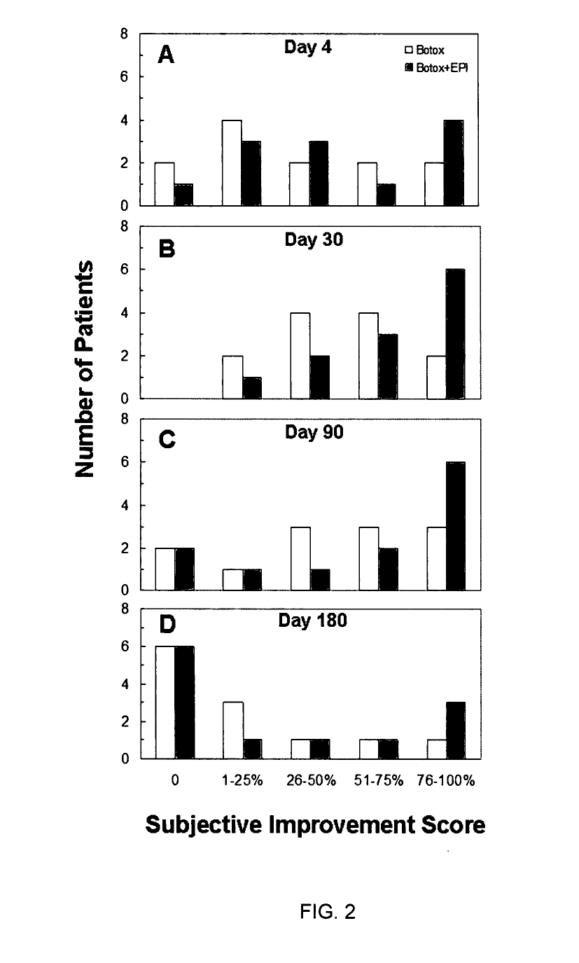

FIG. 2 shows the nominal score data for each treatment across the entire study period. At the 4 day follow-up, FIG. 2A demonstrates that twice the number of patients demonstrated a 76-100% improvement compared to baseline in the Botox plus EPI ( 4/12) versus the Botox alone arm ( 2/12). At the 30 day follow-up, FIG. 2B shows that 6/12 subjects treated with Botox plus EPI versus only 2/12 treated with Botox alone reported a 76-100% improvement representing a 3-fold difference. By 90 days, the effect of Botox alone had peaked with only 3/12 subjects reporting a 76-100% improvement (FIG. 2C). This value remained 50% less than for Botox plus EPI. FIG. 2D shows that by day 180 post-injection, the number of subjects reporting a 76-100% improvement for both treatment arms dropped lower than at 4 days post-treatment (cf. 2A and 2D).]

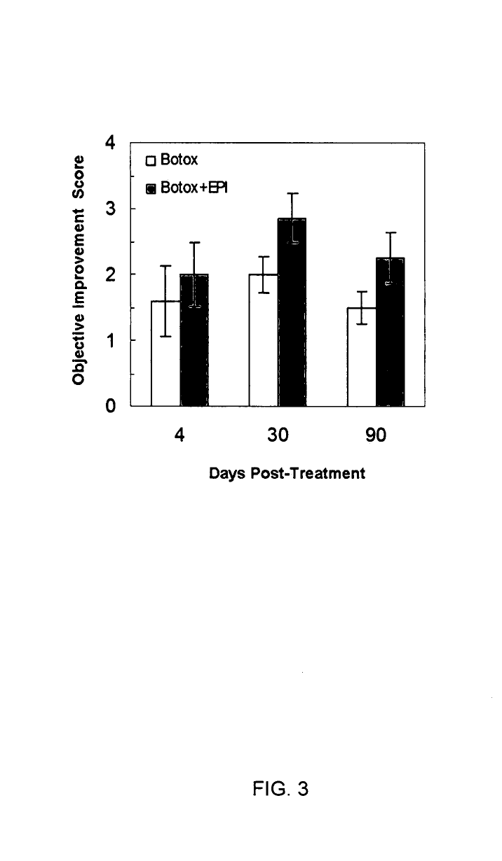

In order to assess whether or not the subjective scores were reliable, digital photographs were examined at 0, 4, 30, and 90 days and objective improvement scores were determined using the same 0-4 nominal scale. FIG. 3 shows by 4 days, a 30% enhancement over the Botox alone treatment (mean 1.6) was noted in the experimental side (mean 2.0); admittedly, this did not reach statistical significance using non-parametric analysis (Mann-Whitney test p=0.09). By 30 days, a statistically significant difference was detected (p<0.05) with an objective score of 2.9 for Botox+EPI compared to 2.0 for Botox alone. At the 90 day mark, both treatments were found to have peaked although Botox+EPI (mean 2.3) maintained a 50% enhancement over Botox alone (mean 1.5).

As one of ordinary skill in the art will appreciate, various changes, substitutions, and alterations could be made or otherwise implemented without departing from the principles of the present invention. Accordingly, the scope of the invention should be determined by the following claims and their legal equivalents.

What is claimed is:

1. A method of increasing the efficacy of treating a muscle or sweat gland hyperactivity condition in a human or mammal with a neurotoxin, comprising:
adding a quantity of a diffusion-limiting agent or an anti-metabolic agent to a neurotoxin;
wherein said quantity is sufficient to increase a therapeutic response of said muscle or sweat gland of said human or mammal to said neurotoxin as compared to said therapeutic response of said muscle or sweat gland of said human or mammal of said neurotoxin without said diffusion-limiting agent or said anti-metabolic agent; and
wherein said adding forms an agent/neurotoxin injection mixture.

2. The method as set forth in claim 1, wherein said adding comprises:
adding a lyophilized form of said diffusion-limiting agent or said anti-metabolic agent to a lyophilized form of said neurotoxin to make said agent/neurotoxin mixture; and
reconstituting said agent/neurotoxin mixture.

3. The method as set forth in claim 1, wherein said adding comprises adding a liquid form of said diffusion-limiting agent or said anti-metabolic agent to a lyophilized form of said neurotoxin.

4. The method as set forth in claim 1, wherein said adding comprises adding a liquid form of said diffusion-limiting agent or said anti-metabolic agent to a liquid form of said neurotoxin.

5. The method as set forth in claim 1, wherein said neurotoxin is a paralytic agent, an anti-hidrotic agent, or a paralytic agent and an anti-hidrotic agent.

6. The method as set forth in claim 5, wherein said neurotoxin is a tetanus toxin, an omega-conotoxin, curare, or a botulinum toxin selected from the group consisting of botulinum toxin type A, B, C, D, E, F, or G.

7. The method as set forth in claim 5, wherein said neurotoxin is aluminum chloride, glycopyrrolate, or robinol.

8. The method as set forth in claim 1, wherein said anti-metabolic agent is a collagen or hydrogel.

9. The method as set forth in claim 8, wherein said collagen is at a concentration in the range of about 0.3 to about 30 mg/ml, and wherein said hydrogel is at a concentration in the range of about 1 to about 30 mg/ml in said injection mixture.

10. The method as set forth in claim 1, wherein said diffusion-limiting agent is a dextran, hyaluronic acid, or epinephrine.

11. The method as set forth in claim 10, wherein said dextran is at a concentration in the range of about 50 mM to about 500 mM, wherein said hyaluronic acid is at a concentration in the range of about 3 to about 100 mg/ml, and said epinephrine is at a concentration in the range of about 1 to about 100 μg/ml in said injection mixture.

12. The method as set forth in claim 1, wherein said hyperactivity condition is facial rhytides or hyperhidrosis.

13. The method as set forth in claim 1, wherein said method decreases side effects, wherein said side effects include but are not limited to eye ptosis or respiratory arrest.

14. A kit for treating a muscle or sweat gland hyperactivity condition in a human or mammal, comprising:
a) a needle;
b) a solvent;
c) a neurotoxin; and
d) a diffusion-limiting agent or an anti-metabolic agent.

15. The kit as set forth in claim 14, wherein said solvent is at least one of preservative-free saline, preservative containing saline, water, or metabisulfite.

16. The kit as set forth in claim 14, wherein said neurotoxin is a paralytic agent, an anti-hidrotic agent, or a paralytic agent and an anti-hidrotic agent.

17. The kit as set forth in claim 14, wherein said neurotoxin is a tetanus toxin, an omega-conotoxin, curare, or a botulinum toxin selected from the group consisting of botulinum toxin type A, B, C, D, E, F, or G.

18. The method as set forth in claim 14, wherein said neurotoxin is aluminum chloride, glycopyrrolate, or robinol.

19. The kit as set forth in claim 14, wherein said neurotoxin, said agent, or both are lyophilized.

20. The kit as set forth in claim 14, wherein said neurotoxin, said agent, or both are in liquid form.

21. The kit as set forth in claim 14, wherein said neurotoxin and said agent are provided separately or are combined in one vial.

22. The kit as set forth in claim 14, wherein said anti-metabolic agent is a collagen or hydrogel.

23. The kit as set forth in claim 22, wherein the amount of said collagen is sufficient to produce an injection concentration in the range of about 0.3 to about 30 mg/ml, and wherein the amount of said hydrogel is sufficient to produce an injection concentration in the range of about 1 to about 30 mg/ml.

24. The method as set forth in claim 14, wherein said diffusion-limiting agent is a dextran, hyaluronic acid, or epinephrine.

25. The method as set forth in claim 21, wherein the amount of said dextran is sufficient to produce an injection concentration in the range of about 50 mM to about 500 mM, wherein wherein the amount of said hyaluranic acid is sufficient to produce an injection concentration in the range of about 3 to about 100 mg/ml, and wherein the amount of said epinephrine is sufficient to produce an injection concentration in the range of about 1 to about 100 μg/ml.
